(19) United States
(12) Patent Application Publication
(10) Pub. No.: US 2023/0110636 A1
(43) Pub. Date: Apr. 13, 2023

(54) Method and device for improving brain function

(71) Applicant: Tianxin Wang, Walnut Creek, CA (US)

(72) Inventor: Tianxin Wang, Walnut Creek, CA (US)

(73) Assignee: Tianxin Wang, Walnut Creek, CA (US)

(21) Appl. No.: 17/985,896

(22) Filed: Nov. 13, 2022

Related U.S. Application Data
(63) Continuation of application No. 16/819,165, filed on Mar. 15, 2020, now abandoned.
(60) Provisional application No. 62/823,656, filed on Mar. 26, 2019.

(51) Int. Cl. A61N 1/36; A61H 39/00; A61N 5/06; A61F 7/00; A61N 1/04

(52) U.S. Cl. CPC A61N 1/36025; A61F 7/007; A61H 39/002; A61N 1/0456; A61N 1/0484; A61N 5/0622; A61F 2007/0008; A61F 2007/0071; A61F 2007/0078; A61H 2201/10; A61H 2201/0207; A61H 2201/0228; A61H 2205/021; A61N 2005/0647; A61N 2005/0651; A61N 2005/0659

(57) ABSTRACT

Methods and therapeutic devices with noninvasive means for improving brain function and treating senile dementia including Alzheimer’s disease are described. The noninvasive means is selected from electrical stimulation, heat stimulation, IR (infrared) radiation or their combinations. In some embodiments, the method and device utilize electrical current passed through acupuncture sites on the human head to achieve desired therapeutical effects.

### CROSS-REFERENCE TO RELATED APPLICATIONS

This application is a continuation of U.S. Application 16/819,165, filed on Mar. 15, 2020, which claims benefit of and priority to U.S. Ser. No. 62/823,656, filed on Mar. 26, 2019, which is incorporated herein by reference in its entirety for all purposes. The entire disclosure of the prior application is considered to be part of the disclosure of the instant application and is hereby incorporated by reference.

### BACKGROUND OF THE INVENTION

Dementia is a broad category of brain diseases that cause a long-term and often gradual decrease in the ability to think and remember that is severe enough to affect daily functioning. Other common symptoms include emotional problems, difficulties with language, and a decrease in motivation. Consciousness is usually not affected. A dementia diagnosis requires a change from a person’s usual mental functioning and a greater decline than one would expect due to aging. The most common type of dementia is Alzheimer’s disease, which makes up 50% to 70% of cases. Other common types include vascular dementia (25%), dementia with Lewy bodies (15%), and frontotemporal dementia. Less common causes include normal pressure hydrocephalus, Parkinson’s disease dementia, syphilis, HIV, and Creutzfeldt-Jakob disease. Diagnosis is usually based on history of the illness and cognitive testing with medical imaging and blood tests used to rule out other possible causes. The mini mental state examination is one commonly used cognitive test. Efforts to prevent dementia include trying to decrease risk factors such as high blood pressure, smoking, diabetes, and obesity.

Currently there is no known cure for dementia. Cholinesterase inhibitors such as donepezil are often used and may be beneficial in mild to moderate disorder. Overall benefit, however, may be minor. There are many measures that can improve the quality of life of people with dementia and their caregivers. Cognitive and behavioral interventions may be appropriate. Educating and providing emotional support to the caregiver is important. Exercise programs may be beneficial with respect to activities of daily living and potentially improve outcomes. Aromatherapy and massage have unclear evidence. Studies support the efficacy and safety of cannabinoids in relieving behavioral and psychological symptoms of dementia. Omega-3 fatty acid supplements from plants or fish sources do not appear to benefit or harm people with mild to moderate Alzheimer’s disease. It is unclear whether taking omega-3 fatty acid supplements can improve other types of dementia. U.S. Pat. Application US20190126062A1 disclosed devices, systems, and methods for treating dementia or Alzheimer’s disease in a subject in need using visual/audio stimuli having a frequency of about 30 Hz to about 50 Hz, and more specifically about 40 Hz, are non-invasively delivered to the subject to entrain gamma oscillations in multiple brain regions of the subject, including the prefrontal cortex (PFC) and the hippocampus.

### SUMMARY OF THE INVENTIONS

This application discloses methods and devices with non-invasive means for improving brain function such as improving sleep and memory, and treating senile dementia including Alzheimer’s disease. The non-invasive means is selected from electrical stimulation, heat stimulation, IR (infrared) radiation or their combinations. In some embodiments, the invention utilizes electrical current passed through acupuncture sites on the human head to achieve the desired therapeutical effects.

In some embodiments the device and method in the current invention is primarily directed towards stimulating one or more of the acupuncture points selected from Baihui (DU/GV20), Sishencong (EX-HN1), Fengchi (GB 20), TaiYang (EX-HN5) and Shenting (DU/GV 24) for the purpose of improving brain function and treating senile dementia including Alzheimer’s disease. Preferably the device in the invention is portable and self-contained with means to be attached to the head. It can be in the form of hat or helmet. The device can produce electrical pulse or heat or IR radiation or their combination and apply them to the said acupuncture points.

In one aspect, the methods and devices described in the current application use electro pulse or electrical pulse in combination with heat or IR radiation applied to acupuncture points or other area in the head to achieve the desired therapeutical effects. A person desiring to improve brain function (e.g. reduce aging, improving memory and sleep, improving blood flow, reducing brain function impairment, reducing cognitive impairment, improving brain tissue growth, improving neuron /nerve integrity, growth, function, activity and function) or treating dementia such as Alzheimer’s disease or vascular dementia places the electrodes on the acupuncture points selected from Baihui, Sishencong, Fengchi, and Shenting and applies electrical stimulation through these electrodes. Heat and/or IR stimulation produced by the device can also be applied to these points for the same purpose. This has the effect of improve brain function and/or reduce symptoms of dementia. The same device and method also be used to treat depression and insomnia. The pulse generating circuitry and power supply can be most conveniently packaged in a housing structure with means to affix to the head such as a hat or helmet shape structure. One or more electrodes are on the inner surface of the housing which is placed in contact with skin. The device is placed so that the electrode/electrodes overlie the acupuncture points known to affect a desired therapy. Alternatively, the device is placed so that the electrodes cloase or overlie a nerve that runs under the acupuncture point, in which case the device may be placed some distance from the associated acupuncture point to provide a comfortable placement for the device. It can also be attached to other areas on the head.

The device can also contain one or more built in heating element that can keep the temperature of the skin contact surface between 40~60 degree to provide a heating effect to the stimulation sites/area. The device can also contain one or more built in IR radiation element that can produce a light and pass it to the skin contact surface to provide a IR stimulation effect to the stimulation sites/area to provide heating effect.

### BRIEF DESCRIPTION OF THE DRAWINGS

FIG. 1 shows an example of a block diagram of the electrical circuitry suitable for the device.

FIG. 2 shows an example of a block diagram of the electrical circuitry suitable for the device with additional heating elements for the current invention.

FIG. 3 illustrates an example of the circuit diagram suitable for the device of the current invention.

FIG. 4 shows an example of the device electrode assembly.

FIG. 5 shows an example of another type of device electrode assembly.

FIG. 6 shows an example of a device electrode assembly with heating element.

FIG. 7 shows an example of a device electrode assembly with light radiation element.

FIG. 8 shows examples of electrode assembly with IR radiation and heating function.

FIG. 9 is a perspective illustration of an example of the device of the invention as worn on the head of a person using the device.

FIG. 10 shows examples of stimulation points on the head.

FIG. 11 shows an example of device containing an array of electrode and heating element.

### DETAILED DESCRIPTION

In some embodiments, the preferred (but not limited to) electrical pulse frequency used bt the method and device of the current invention is between 2~100 Hz or the combinations of the frequency within 2~100 Hz; the preferred pulse width is 0.001~ 10 ms and the preferred current is 0.5-100 milliamperes. In some embodiments, exemplary preferred electrical pulse repetition rate of approximately 2 pulses per second with a pulse width of 600 microseconds, or approximately 15 pulses per second with a pulse width of 300 microseconds, or approximately 40 pulses per second (e.g. 30-50 or 35-45 pulses) with a pulse width of 200 microseconds, or approximately 100 pulses per second with a pulse width of 200 microseconds, or the combination of the above wave patterns can provide effective result. The preferred electrical power levels can be about 0.5-50 milli-amps peak pulse height or other power level that the subject in need feel comfortable. A wider range of pulse patterns can also be used in the non-invasive nerve stimulation devices. Bi-directional pulse is preferred to avoid skin damage. In general, 10- 60 min stimulation daily or on demand can provide effective treatment to the person in need.

In other examples, medium-frequency currents are used. Typical medium frequency (e.g. 1~10 kHz) can stimulate the muscle and provide a massage like feeling to the area treated. In order to interrupt the current after each depolarization, rhythmically increasing and decreasing the amplitude (amplitude modulation) can be applied. The Amplitude Modulation Frequency (AMF) determines the frequency of the depolarization. The AMF corresponds to the frequencies used in low-frequency electrotherapy. The amplitude modulation can also be replaced by pulse width modulation.

The electric current pattern used in modulated medium frequency electrotherapy can also be used in the devices of the current invention. Suitable frequency can be 2-10 kHz. The combination of low frequency and medium frequency can be used, e.g. the combination of frequency of 1~150 Hz low frequency current and 2~10 kHz medium frequency current. The low and/or medium frequency can have varieties of wave shape/pattern (e.g. sinusoidal wave, squire wave, continued, discontinued, varying amplitude, symmetrical wave or unsymmetrical wave etc.). The amplitude of medium frequency can vary at the low frequency pattern. In some examples, suitable current is between 0.05~ 5 mA.

A gamma wave is a pattern of neural oscillation in humans with a frequency between 25 and 100 Hz, though 40 Hz is typical. It can benefit and improve brain function. Electric frequency that can stimulate gamma wave in brain can be used for the current invention, for example between 25-100 HZ, preferably between 30~60 Hz, or 35~50 Hz. In some embodiments, the electrical pulse frequency is between 35~45 Hz. In some embodiments, the electrical pulse frequency is 40 Hz. In some embodiments, the frequency is 40 Hz and the peak width is between 0.1~ 0.6 ms, e.g. 0.3 ms. In some embodiments, the electrical pulse is applied in cycled batch, in each cycled batch the current start from low to high and then keep constant for certain time such as 5-10 s, then drop to low gradually for the next cycled batch as shown in the figure below. There could be an interval period (no electric pulse) between each cycled batch, e.g. 2-10 seconds. The combination of different frequency of gamma wave can also be used, for example the frequency is 35 Hz in the first 5 min and then 40 Hz for 5 min and then 45 Hz for 5 min and then goes back to 40 Hz for 10 min.

The device can also contain one or more built in heating means that can keep the temperature of the skin contact surface between 35~60 degree to provide a heating effect to the stimulation sites/area. The heating means can be an electrical heating element powered by a battery. The preferred heating temperature at the surface is between 35~55 degree. A preferred heating element is self-regulating heater such as polymer PTC heating elements or ceramic PTC heater. Resistive heaters can be made of conducting PTC rubber materials or ceramic PTC material where the resistivity increases exponentially with increasing temperature. Such a heater will produce high power when it is cold, and rapidly heat up itself to a constant temperature. Due to the exponentially increasing resistivity, the heater can never heat itself to warmer than this set temperature. The temperature can be chosen during the production of the rubber or ceramic. The heating step can be cycled to avoid skin desensitization. For example, each heating step is 1-10 min followed by 1-3 min none heating period.

The device can also contain one or more built in IR radiation element that can produce IR radiation and pass it to the skin contact surface to provide a photon stimulation and heating effect to the stimulation sites/area. The IR radiation elements can be tungsten wire, carbon, or alloys of iron, chromium, and aluminum as well as LED and laser or their combinations to provide broader wavelength coverage. IR radiation elements are widely used in physical therapy and they can be adapted readily for the current invention. The device can have one centralized IR radiation elements connected to multiple infrared optical fibers, which can transfer the IR radiation to the desired stimulation sites. The surface of the housing can have one or more light transparent windows to allow the photon emitted from the light source reaching the skin. Suitable wavelength of IR is between 700 nmm-1 mm. It can be either NIR or MIR or FIR or its combination. In some embodiments, it is between 800 nm ~ 100 um. In other examples, it is between 1 um~ 20 um. The output power of IR radiation can be adjusted to proved effective and safe radiation. Preferably the suitable power level can heat the target skin site to 40° C.~50° C. and does not burn/damage the skin and is acceptable by the user. The radiation can be either continuous or pulsed as those used in the electric pulse stimulation described previously. In one example, a 10 W IR radiation element with 2 µm-10 µm wavelength output is used; the output is coupled to IR optical fibers to deliver IR radiation to the treatment sites. In another example, a 808 nm GaAlAs laser or 1550 nm laser can be used, connected to multiple optical fiber to deliver IR pulse to the sites and the gamma wave frequency such as 30-50 Hz at the power level of 10 -1000 mW/cm² can be applied. When IR LED is used, each stimulation site can have its own IR LED attached without the need of using optical fibers.

Therefore, the current invention discloses a method for improving brain function in a subject in need thereof, the method comprising administering a non-invasive electrical stimulus have a frequency of about 35 Hz to about 45 Hz to the subject’s surface area on the head. Additional heat stimulation can also be is applied to the head.

The device can have a communication module that can communicate with an external control (command) module to receive commands of the stimulation output (e.g. stimulation type, time, frequency, on/off, power level and pulse pattern) and produce stimulation accordingly. The external control module can be a remote or computer or a cell phone with dedicated application installed. The communication can utilize Wi-Fi or blue tooth or IR or radio signal. The device can have an on/off control to turn on or turn off the communication module.

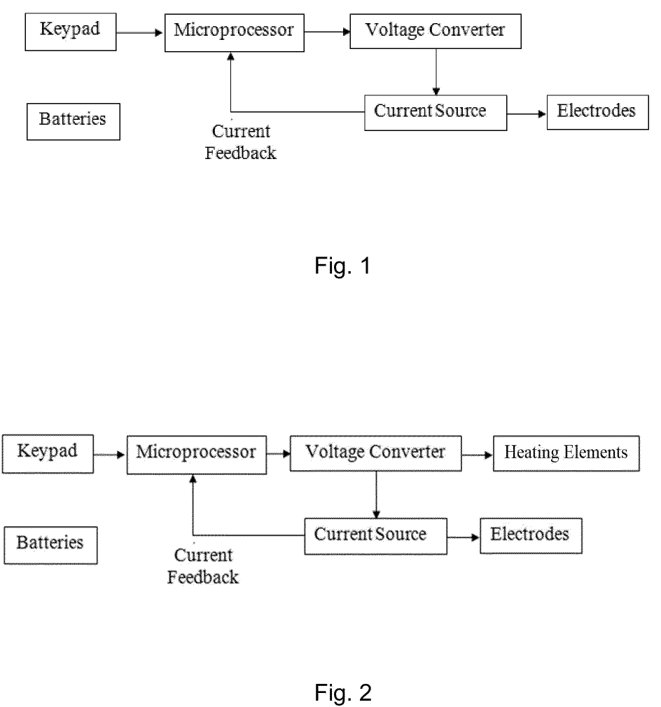

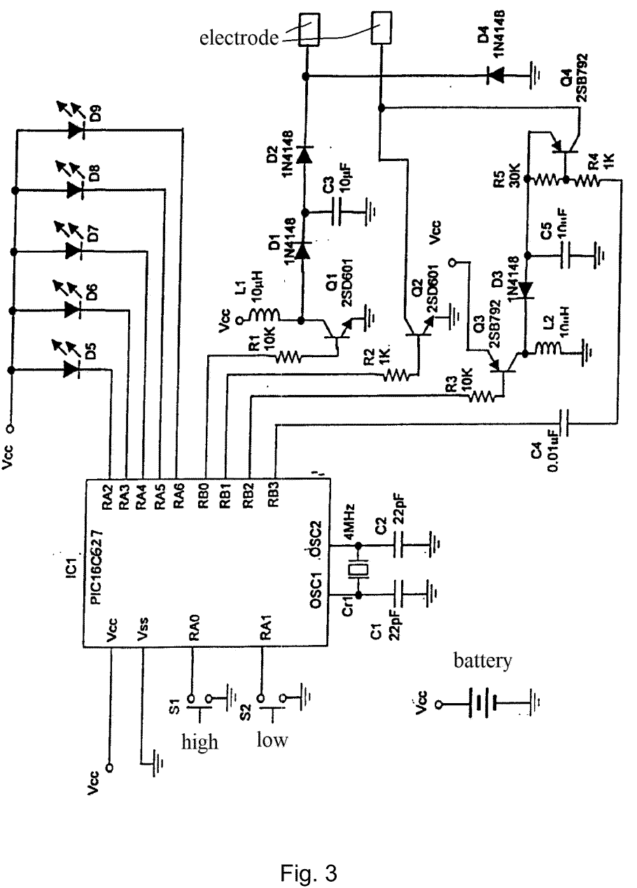

The electrical circuitry of the device can be implemented with well-known art. There are many designs to implement the circuitry. FIG. 1 illustrates an example of the circuit diagram suitable for the device. The microprocessor receives input from the keypad on the device to set the intensity level of the stimulate pulses, and current feedback to regulate the microprocessor output to voltage converter and current source to generate the stimulate pulses at the current level set by the keypad. The voltage converter converts battery voltage to high voltage. The output of the current source is connected to the electrodes, which contact skin. The batteries such as a rechargeable battery pack supply power to every block. FIG. 2 illustrates an example of the circuit diagram suitable for the device with additional heating elements controlled by microprocessor and voltage converter. FIG. 3 illustrates an example of the circuit diagram suitable for the device.

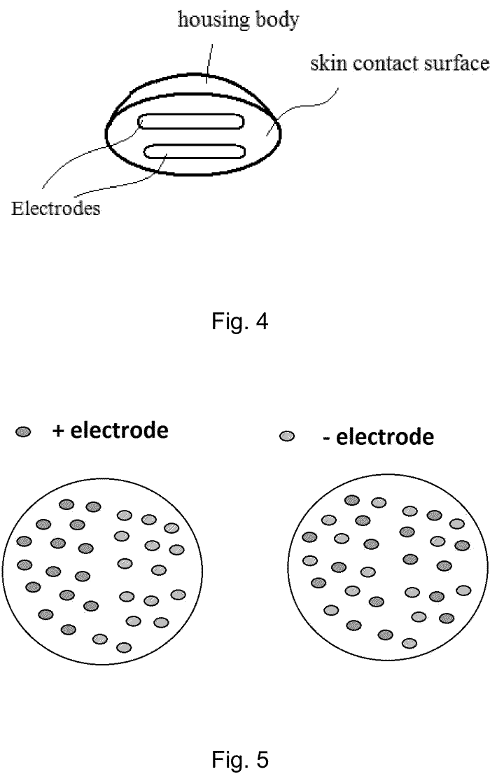

FIG. 4 shows one embodiment of the device electrode assembly. On the skin-contacting surface of the device body, two electrodes are aligned in a side by side manner. One electrode will function as cathode (or connects to the hot wire of the current source) and another electrode will function as anode (or connects to the neutral wire). External electrode can also be used to be connected with the housing via connection means such as wire to deliver the electrical pulse to the external electrode. If the device contains multiple housing bodies, each house body can contain only one electrode; the electrode in some housing function as cathode and the electrode in other housing function anode and they can change polarity during stimulation.

FIG. 5 shows skin-contacting surface of an embodiment of another type of device electrode assembly. On the skin-contacting surface of the device body, multiple small electrodes are attached. Some electrodes function as cathodes and others function as anodes.

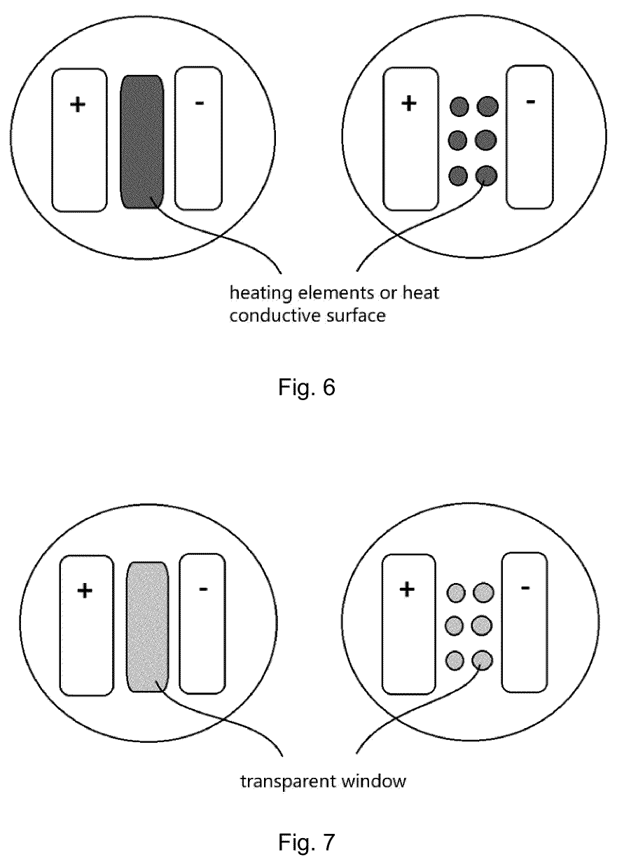

FIG. 6 shows one embodiment of a device electrode assembly with heating element. As shown in the figure, on the skin-contacting surface of the device body, two electrodes are attached. Between the two electrodes is a heating element or a heat conducting area (e.g. a metal piece) with a heating element attached beneath. Alternatively, the heating element or the heat conductive area can be in an array form. Yet in another alternative, the heat conductive area can also be the electrode.

FIG. 7 shows one embodiment of a device electrode assembly with light radiation element. As shown in the figure, on the skin-contacting surface of the device body, two electrodes are attached. Between the two electrodes is a transparent window with a laser output source attached beneath. Alternatively, the transparent window can be in an array form. A laser light source delivers the light to these windows with optical fibers.

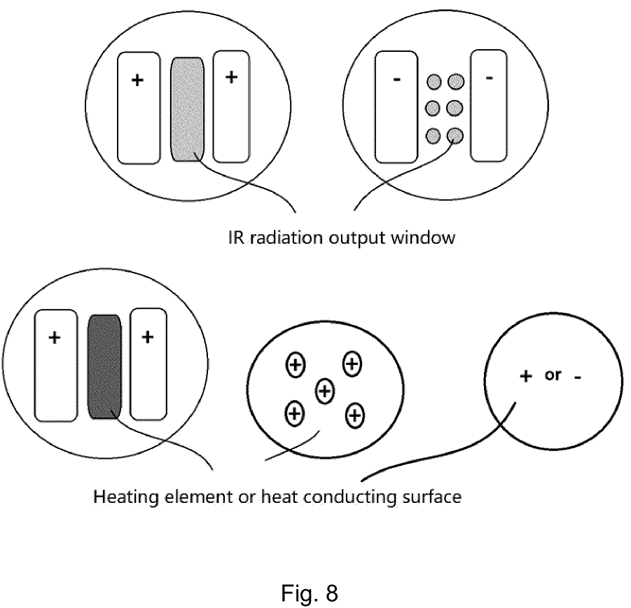

Because multiple stimulating sites are used, some electrode assembly function as cathode and others function as anode, therefore no need to have different type of electrode (cathode or anode) integrated in one electrode assembly. Examples are shown in FIG. 8. The electrodes can be either cathode or anode or switch alternatingly.

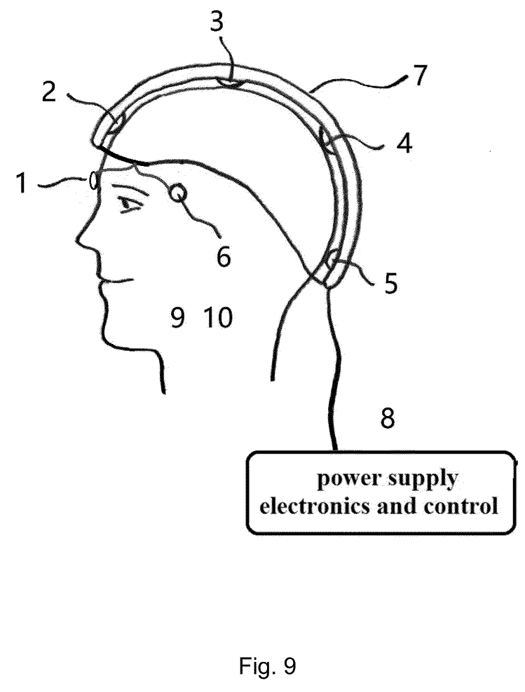

FIG. 9 is a perspective illustration of one embodiment of the device of the invention as worn on the head of a person using the device. The power supply and pulse generating circuitry for the device can be integrated within a helmet or hat like housing 7. Alternatively, the control, power supply and/or pulse generating circuitry can also be placed in another housing 8 which is connected with the housing 7 with wires as shown in FIG. 9. The housing can also be in other shape and format such as a head band, as long as it contains means to affix it on the head or provide a support to place the housing on the head. An external control unit 8 is connected to the housing in the drawings, having stimulating power level control buttons and one LCD status display. The power level control buttons control the output power level of the electrodes. Higher power level (the intensity of the stimulation current) generally gives stronger stimulation , which may generate better therapeutic effects. The patients use the control buttons to adjust the power level to achieve desired therapeutic effects and the best comfort. These buttons may also be used to control the pulse patterns. The LCD display displays the working status of the device such as the current power level and pulse pattern. A timing function can also be incorporated within the LCD.

Four electrode assemblies 2, 3, 4 and 5 are aligned at the inner surface of the housing, allowing them to contact the skin at ShenTing, Baihui, HouDing and FengFu acupuncture sites respectively. Two external electrode assemblies 1 and 6 (such as those described in FIG. 4) are connected to the housing 7 with wire to get electricity and are attached to the sites TaiYang and YinTang sites respectively to provide stimulation. The housing sends out electrical pulse to the electrode assembly to apply electrical stimulation to the person in need. Self-adhesive electrodes could be used in external electrode assembly. A rigid or semi rigid connection structure containing the electric wire can also be used to link the external electrode assembly with the housing besides using wires.

The device in FIG. 9 can also contains means to provide heating and IR radiation to these acupuncture sites as previously described. The heating/IR stimulation can be applied at the same time as electric stimulation or be applied alternately or sequentially. In some embodiments the device contains heating or IR radiation means only without any eclectic stimulating means and the therapeutic effect is achieved by direct heating or IR radiation only.

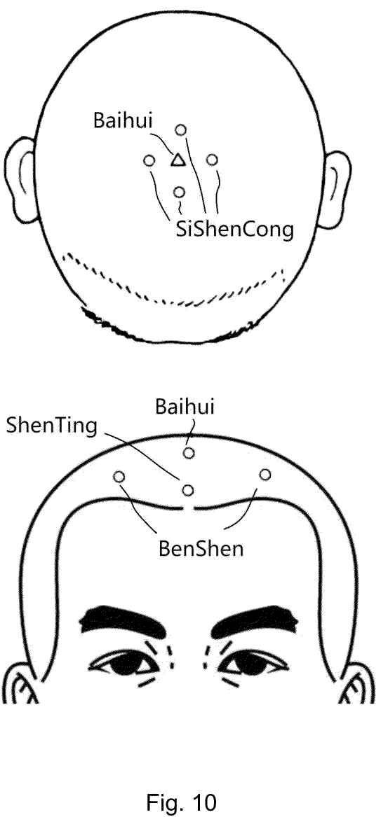

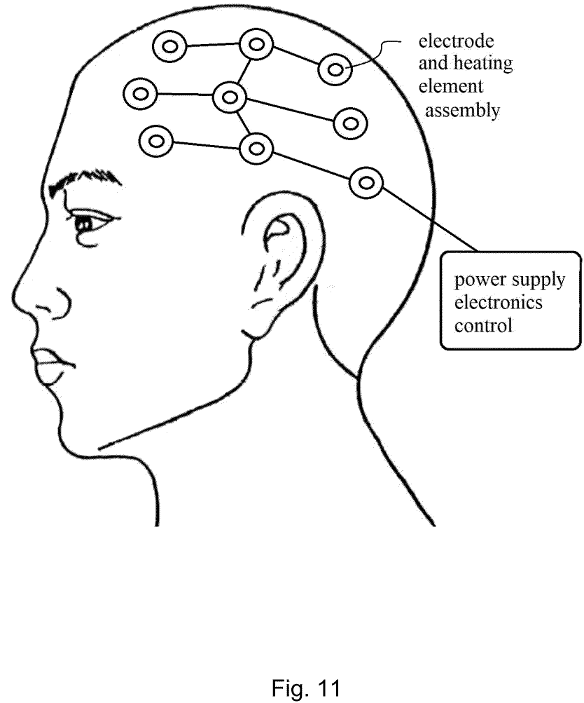

As shown in FIG. 9, a person in need places the device on his head and attaches the electrodes on the selected acupuncture points to apply electrical stimulation to these points through these electrodes. Heating or IR radiation stimulation can also be applied at the same time or separately to these sites. The primary stimulation points (some are shown in FIG. 10) are selected from Baihui, Sishencong, Fengchi, Shenting, Benshen, Shangxing, Fengfu,Yintang, Taiyang, Yamen, Dangyang, Dingshen, Shoumian, Chengling and Naohu acupuncture sites. One or more primary stimulation points can be used. Preferred adjunctive acupuncture points can be selected from Dazhui, NeiGuan, SanYinJiao, Renzhong, Shenmen, Xinyu, Jianshi, Guanyuan, Yongquan, Taixi, Xuehai, Shuangling, Wangu, Dashu, Renying and Shenyu acupuncture sites. Using adjunctive acupuncture points in combination with primary stimulation points gives higher effectiveness than using the primary acupuncture points alone. In addition, stimulating some of these points requires attaching the electrode in places other than the head (e.g. electrode need to be attached to the wrist to stimulate NeiGuan point). In order to stimulate adjunctive acupuncture points, external electrodes connected with the main housing (e.g. the housing on the head or the external power supply/electronics) can be used to reach these sites. The stimulation at each point/area can be simultaneously or sequentially based on a predefined pattern (e.g. cycle from left to right or from center to peripheral and etc.). As described previously, the electrical stimulation can be replaced with heating or IR radiation with the corresponding means.

Alternatively, the area for stimulation is limited to acupuncture sites. It can be placed on other areas on the head as long as the subject feel comfortable. In some embodiments, the device as shown in FIG. 10 can be an array of electrode or an array of electrode with heating/IR radiation means covering the skin on top of the brain with the stimulation pulse frequency between 25~100 Hz, preferably between 35~45 Hz. For example, a device used by Novocure can be modified to give electrical stimulation with a frequency of 40 Hz for improving brain function and treating senile dementia including Alzheimer’s disease. The device disclosed in U.S. Pat. US8715203 and US7805201 can be modified to have the desired frequency for the current invention.

In one example, a device described in FIG. 9 is attached to a volunteer’s head to stimulate the acupuncture sites selected from YinTang, ShenTing, BaiHui, TaiYang and SiShengCong with electrical pulse of 40 Hz at the power level of the volunteer feels comfortable for ~0.5 hour every day for 2 weeks. The volunteer feels improved memory, sleep quality and cognitive activity.

In another example, a device is attached to a volunteer’s head to stimulate ShenTing, BaiHui, TaiYang and SiShengCong sites with electrical pulse of 40 Hz at the power level of the volunteer feels comfortable for ~0.5 hour every day for 2 weeks. The volunteer feels improved memory, sleep quality and cognitive activity.

In another example, a device containing an array of electrodes is attached to a volunteer’s head to stimulate the top surface of head at non-acupuncture sites with electrical pulse of 35-45 Hz at the power level of the volunteer feels comfortable for ~1 hour every day for 2 weeks. The volunteer feels improved memory, sleep quality and cognitive activity.

In another example, a device further containing heating elements is attached to a volunteer’s head to stimulate the acupuncture sites selected from YinTang, BenShen, HouDing, FengDu and FengChi with electrical pulse of 35-45 Hz at the power level of the volunteer feels comfortable and heat stimulation for 0.5-1 hour every day for 2 weeks. The volunteer feels improved memory, sleep quality and cognitive activity.

Furthermore, a device with shape similar to that in FIG. 9 containing means to provide direct heating (e.g. using heating element) and/or IR radiation to the head, especially the top and backside of the head can also be used to improve brain function and/or reduce symptoms of dementia, by improving the blood flow in the brain. Higher temperature (heating) to the brain area can boost the blood flow, therefore improve brain function and/or reduce symptoms of dementia. This device is essentially a hat or helmet with build in heating means that can cover most of the area on the top and/or back of the head, not limited to the acupuncture sites previously described. It can have an insulating layer to maintain the high temperature inside the covered area (e.g. 40-60C°). The heating can be constant or intermittent. Person in need can put it on to heat their brain area to improve brain function and/or reduce symptoms of dementia (e.g. 0.5 hr 3 times a day). The heating/IR stimulation can be applied at the same time as electric stimulation or be applied alternately or sequentially. In some embodiments the device contains heating or IR radiation means only without any eclectic stimulating means and the therapeutic effect is achieved by direct heating or IR radiation only. The device can also have an attachment with built in direct heating/IR radiation means that can wrap the neck of the person in need to heat their blood vessel in the neck to boost the blood flow, which can be essentially a muffler/scarf with built in direct heating/IR radiation means. The current invention discloses a method to improve brain function and/or reduce symptoms of dementia by increase the blood flow in the brain, which is achieved by applying direct heating/IR radiation to the head and neck of the person in need.. The IR radiation can also be given at the frequency of 35~45 Hz.

The device suitable for the current invention can be in many different format and shape such as in the form of hat or hairband or glass or helmet or a self-stick patch form. Their skin contacting portion contains one or more electrodes that can be used to apply electric pulse to the subject in need. They can have means to commutate with a remote controlling device such as cell phone with either Bluetooth or Wi-Fi or other radio frequency signals and be controlled by the remote controlling device. A cell phone application can be used to control the device.

1. A method for improving memory in a human subject in need thereof, the method comprising:
administering a heat stimulation to at least one of the subject’s head skin surface with a heating means having a skin contacting surface with the surface temperature maintained between 35-55 degrees; and
administering a non-invasive electrical stimulation having a frequency of about 35 Hz to about 45 Hz to the subject’s TaiYang acupuncture site using an electrode assembly attached to the TaiYang site, wherein said each electrode assembly has a skin contacting surface and at least a cathode and an anode on its skin contacting surface.

2. The method of claim 1, wherein the frequency is of about 40 Hz.

3. The method of claim 1, further comprising administering a non-invasive electrical stimulation having a frequency of about 35 Hz to about 45 Hz to the subject’s Baihui, Sishencong, and Shenting sites.

4. A method for improving sleep quality in a human subject in need thereof, the method comprising:
administering a heat stimulation to at least one of the subject’s head skin surface with a heating means having a skin contacting surface with the surface temperature maintained between 35-55 degrees; and
administering a non-invasive electrical stimulation having a frequency of about 35 Hz to about 45 Hz to the subject’s TaiYang acupuncture site using an electrode assembly attached to the TaiYang site, wherein said each electrode assembly has a skin contacting surface and at least a cathode and an anode on its skin contacting surface.

5. The method of claim 4, wherein the frequency is of about 40 Hz.

6. The method of claim 4, further comprising administering a non-invasive electrical stimulation having a frequency of about 35 Hz to about 45 Hz to the subject’s Baihui, Sishencong, and Shenting sites.

7. A device for improving memory and sleep quality in a human subject in need thereof, said device comprising:
a small housing having a size and shape adapted to be worn on the human’s head;
one or more heating means having a skin contacting surface with the surface temperature maintained between 35-55 degrees; and
circuit means for generating pulsed electrical stimulation signals of selected amplitude and cycle rate at a frequency of about 35 Hz to about 45 Hz and delivering the electrical stimulation signals to at least one external electrode assembly, where in the electrode assembly is connected to said circuit means with wire and each said electrode assembly has at least a pair of cathode and anode on its skin contacting surface.

8. The device of claim 7, wherein the cathode and anode can change polarity during operation.

9. The device of claim 7, wherein the electrode assembly is attached to the TaiYang acupuncture site of said subject.
